(19) United States
(12) Patent Application Publication
(10) Pub. No.: US 2010/0049302 A1
(43) Pub. Date: Feb. 25, 2010

(54) STENT FOR EXPENDING INTRA LUMINAL

(76) Inventors: Sung-Gwon Kang, Gyunggi-Do (KR); Seung-Hwan Jaegal, Seoul (KR); Woo-Sung Yang, Seoul (KR); Soo-Ho Lee, Seoul (KR); Se-Chul Lee, Seoul (KR)

Correspondence Address: BIRCH STEWART KOLASCH & BIRCH, PO BOX 747, FALLS CHURCH, VA, 22040-0747, US

(21) Appl. No.: 12/531,262

(22) PCT Filed: Oct. 30, 2007
(86) PCT No.: PCT/KR07/05388

§ 371 Date: Sep. 14, 2009

(30) Foreign Application Priority Data
Mar. 14, 2007 (KR) 10-2007-0024747

(51) Int. Cl. A61F 2/06

(52) U.S. Cl. 623/1.15

(57) ABSTRACT

The present invention relates to a stent for intraluminal expansion. The stent for intraluminal expansion comprises an inner stent A, an outer stent B and fixing threads C for fixing these stents as one unit. The outer stent B is inserted over the inner stent A in such a way that the space portions of the inner stent A and the space portions of the outer stent B are alternated with each other, so the outer surface of the inner stent and the inner surface of the outer stent are in close contact with each other, and both ends of the outer stent and inner stent are fixed as one unit by fixing threads C. According to the stent for intraluminal expansion of the present invention, in which form recoverability of each of the inner stent A and the outer stent B is excellent, expansion is easy after it is inserted into the lumen body because the cylindrical film membrane is not fixed by fixing thread C, and the space portions Id of the inner and outer stents are overlapped alternately with each other so that it is possible to effectively prevent cancer cells, etc. from penetrating into the lumen.

### TECHNICAL FIELD

The present invention relates to a stent for intraluminal expansion, more specifically to a stent for intraluminal expansion, which excels in form recoverability and expansibility after it is inserted into the lumen, and the space portions of the inner and outer stents are alternately overlapped so as to effectively prevent cancer cells from penetrating into the lumen.

### BACKGROUND ART

In general, lumens in the human body can become stenosed by diseases occurring in the human body, so that the function is lowered or no functions are possible in serious cases. For example, the esophagus is stenosed due to esophageal cancer, smooth blood circulation is not possible due to arteriosclerosis, or the track for bile from liver to flow is stenosed.

In such cases, the stenosed lumen should be expanded or the expanded lumen should be prevented from becoming narrow again. As a method for expanding the stenosed passageway and maintaining it in such a case, there is a method of inserting a so-called stent into the lumen.

Normally, as a stent for intraluminal expansion, a cylindrical stent woven with shape memory alloy so as to have a plurality of space portions is generally used.

It is preferable to manufacture stents for intraluminal expansion with the unit sizes of the stent space portions made small to effectively prevent blood, etc. from penetrating into the lumen. But there is a limit to manufacturing with the unit size of space portion below a certain level in terms of manufacturing technology as much time and effort are required.

Also, the stent for intraluminal expansion requires expansibility for good expansion after insertion into the lumen and form recoverability.

As a conventional stent for intraluminal expansion, a stent for intraluminal expansion disclosed in Korean Patent Laid-Open No. 2000-002967 is known to the general public. The stent for intraluminal expansion described in this document comprises a spiral stent member in which filaments are spirally wound so as to make a cylindrical shape having a certain diameter; a zigzag stent member in which filaments are wound in zigzags so as to have a diameter larger than the spiral stent member and make a cylindrical shape; a first cover member which is wrapped on the inner and outer surfaces of the spiral stent member; a second cover member which is wrapped on the inner and outer surfaces of the zigzag stent member; and a connecting member for connecting the first cover member and the second cover member.

The above mentioned conventional stent can be reduced to have a smaller diameter as it has excellent flexibility due to the spiral stent, and it excels in expansibility for expanding the lumen that is narrowed due to the zigzag stent, and in stability so that it is not easily moved from the transplanted portion of the lumen. However, it has disadvantages that expansibility is lowered after it is inserted into the lumen since the first cover member and the second cover member are connected as one body within the connected portion, that there is a limit to making the unit size of each space portion of the stent small to prevent cancer cells, etc. from penetrating into the lumen, and that a lot of time arid effort are required for use it.

In addition, as another conventional stent for intraluminal expansion, a stent for intraluminal expansion is disclosed in Korean Patent Registration No. 0561713. In the stent for intraluminal expansion described in this patent, an artificial tube is inserted in close contact with the outer circumference of a cylindrical inner stent that is woven with wire so as to contain rhombic space portions, and an outer stent that has the same structure as the inner stent is inserted in close contact with the outer circumference of the artificial tube, and all of said inner stent, artificial tube and outer stent are fixed as one body by fixing threads.

The above mentioned conventional stent for intraluminal expansion excels in flexibility and contractibility since it contains rhombic space portions in the structure. But it has problems that it is bad at form recoverability for recovering and maintaining the original form as it is, that expansibility is lowered after it is inserted into the lumen because the artificial tube is fixed as one body with the inner stent and outer stent, and that because the inner stent and outer stent are combined in such a way that the space portions align with each other, the unit sizes of the space portions are not reduced due to combination of these, so it is not possible to effectively prevent cancer cells, etc. from penetrating into the lumen.

As yet another conventional stent for intraluminal expansion, a stent for intraluminal expansion disclosed in Korean Patent Registration No. 0448329 is known to the general public. The stent for intraluminal expansion described in this document comprises a cylindrical body woven with shape memory alloy so as to have a plurality of space portions, an expanded portion with the diameter expanded outwardly at both ends of the body, and a film coated on the inner surfaces of the body and the expanded portion, wherein a cylindrical auxiliary body woven with shape memory alloy is installed on the outer of the body, one side of the auxiliary body is adhered to the body, and space in which cells to be bred are filled is formed between the body and the auxiliary body.

The above mentioned conventional stent for intraluminal expansion has excellent flexibility since it is of a spiral structure. But it is bad at form recoverability for recovering and maintaining the original form as it is, and because the space portions of the body (inner stent) and auxiliary body (outer stent) are combined so as to align with each other, the unit sizes of the space portions are not reduced due to combination of these, so it has a problem that it is not possible to effectively prevent cancer cells, etc. from penetrating into the lumen.

### DETAILED DESCRIPTION OF THE INVENTION

### Problem to be Solved by the Invention

Therefore, it is an object of the present invention to provide a stent for intraluminal expansion, in which form recoverability of each of the inner stent A and the outer stent B is excellent, expansion is easy after it is inserted into the lumen body because the cylindrical film membrane is not fixed by fixing thread C, and the space portions id of the inner and outer stents are overlapped alternately with each other so that it is possible to effectively prevent cancer cells, etc. from penetrating into the lumen.

### Technical Solution

To achieve the above object, there is provided a stent for intraluminal expansion comprising an inner stent A, an outer stent B and fixing threads C for fixing these stents as one unit, wherein said outer stent B is inserted over said inner stent A in such a way that the space portions of said inner stent A and the space portions of said outer stent B are alternated with each other, so the outer surface of said inner stent and the inner surface of said outer stent are in close contact with each other, and both ends of said outer stent and inner stent are fixed as one unit by fixing threads C.

### Advantageous Effects

According to the stent for intraluminal expansion of the present invention, in which form recoverability of each of the inner stent A and the outer stent B is excellent, expansion is easy after it is inserted into the lumen body because the cylindrical film membrane is not fixed by fixing thread C, and the space portions id of the inner and outer stents are overlapped alternately with each other so that it is possible to effectively prevent cancer cells, etc. from penetrating into the lumen.

### BRIEF DESCRIPTION OF THE DRAWINGS

This and other objects, features, aspects, and advantages of preferred embodiments of the present invention will be more fully described in the following detailed description, taken in conjunction with the accompanying drawings. In the drawings:

FIG. 1 and FIG. 2 are photographs showing the stents for intraluminal expansion according to the present invention;

FIG. 3 is a schematic view showing the structure of the inner and outer stents composing the present invention;

FIG. 4 is a schematic view showing the stent for intraluminal expansion of the present invention as seen in a state in which the outer stent B is inserted in the inner stent A, and both ends of these are fixed by fixing threads C;

FIG. 5(a) and (b) are schematic views showing the manufacturing steps of the inner stent and outer stent composing the stent for intraluminal expansion of the present invention; and

FIG. 6 is a photograph showing by magnifying a part of the stent for intraluminal expansion of the present invention as seen in a state in which the outer stent B is inserted in the inner stent A, and both ends of these are fixed by fixing threads C.

### DESCRIPTION OF SYMBOLS FOR MAJOR PARTS IN DRAWINGS

| | |
|---|---|
| A: inner stent | B: outer stent |
| C: fixing thread | 1: first shape memory alloy wire |
| 2: second shape memory alloy wire | |
| 1a: straight line portion | 1b: peak portion |
| 1c: valley portion | 1d: space portion |

### BEST MODE FOR CARRYING OUT THE INVENTION

Below will be described in detail a stent for intraluminal expansion of the present invention with reference to the accompanying drawings.

The stent for intraluminal expansion of the present invention comprises, as shown in FIG. 4, a cylindrical inner stent A and outer stent B woven by wires 1 and 2 of shape memory alloy so as to have a plurality of space portions 1d, and fixing threads C for fixing these stents as one unit. Since the outer stent B is inserted over the inner stent A so that the space portions of the inner stent A and the space portions of the outer stent B alternate with each other, the outer surface of the inner stent and the inner surface of the outer stent are in close contact with each other.

Both ends of the inner stent and the outer stent are fixed as one unit by fixing threads C.

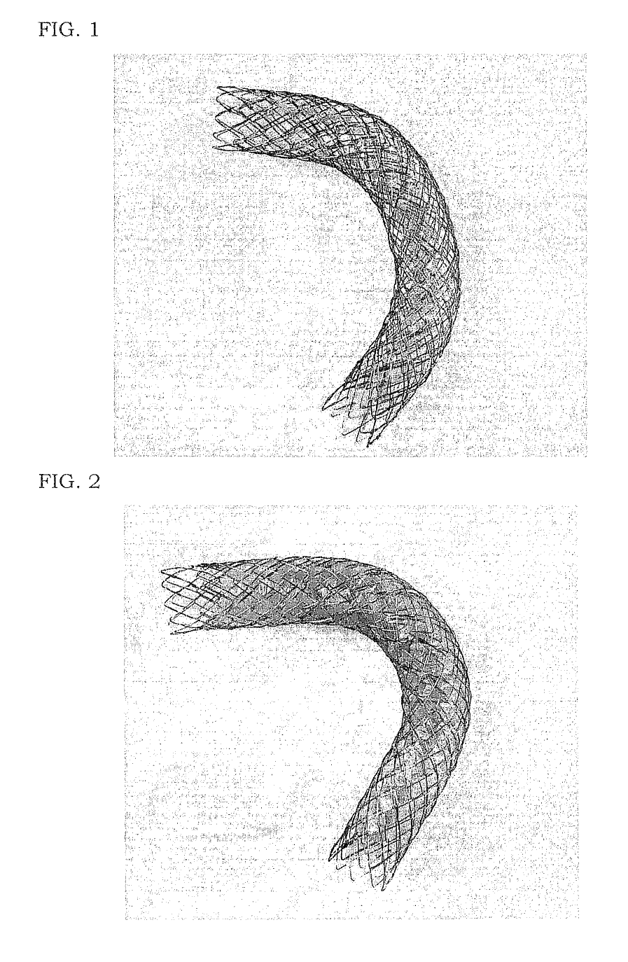

To describe in more detail the stent for intraluminal expansion of the present invention having such characteristics, it comprises a cylindrical inner stent A and the outer stent B woven by wires 1 and 2 of shape memory alloy, and fixing threads C for fixing these stents, as shown in FIG. 1 and FIG. 4.

FIG. 1 is a photograph of a stent (without cylindrical film membrane) for intraluminal expansion according to the present invention and FIG. 4 is a schematic view of the stent for intraluminal expansion.

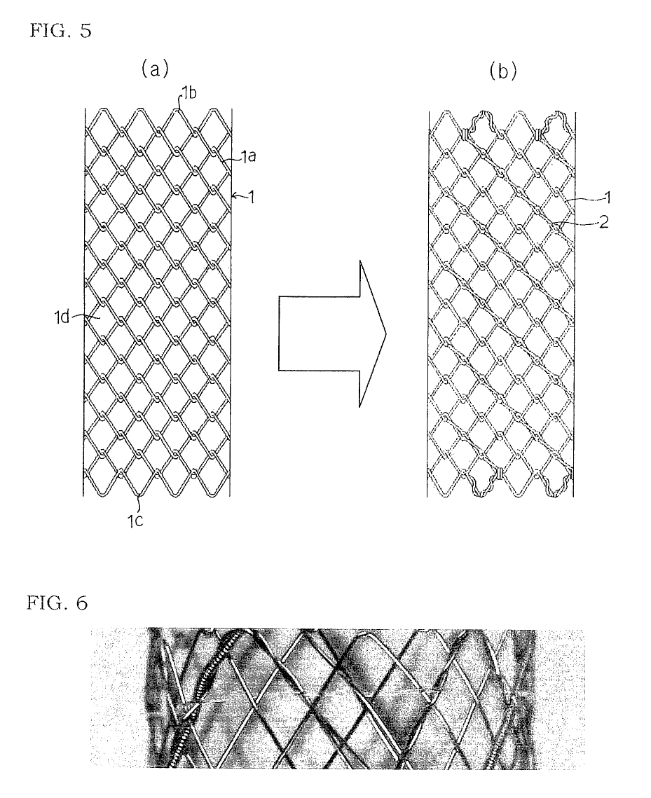

FIG. 6 is a magnified photograph of the portion in which the inner stent A and the outer stent B are fixed as one unit by fixing threads C.

The present invention discloses the stent for intraluminal expansion in which a cylindrical film membrane is additionally inserted between inner stent A and outer stent B, as shown in FIG. 2.

FIG. 2 is a photograph of a stent for intraluminal expansion of the present invention which additionally contains a cylindrical film membrane.

The above mentioned film membrane is composed of silicon resin, etc. and the material of cylindrical film membrane is not specially limited in the present invention. If the cylindrical film membrane is additionally inserted between inner stent A and outer stent B, a state in which both ends of the cylindrical film membrane are not fixed as one unit with both ends of the inner stent A and the outer stent B is maintained by fixing threads C, so that after the stent for intraluminal expansion is inserted it can be expanded quickly.

In other words, only both of the ends of inner stent A and outer stent B are fixed as one unit by fixing threads C.

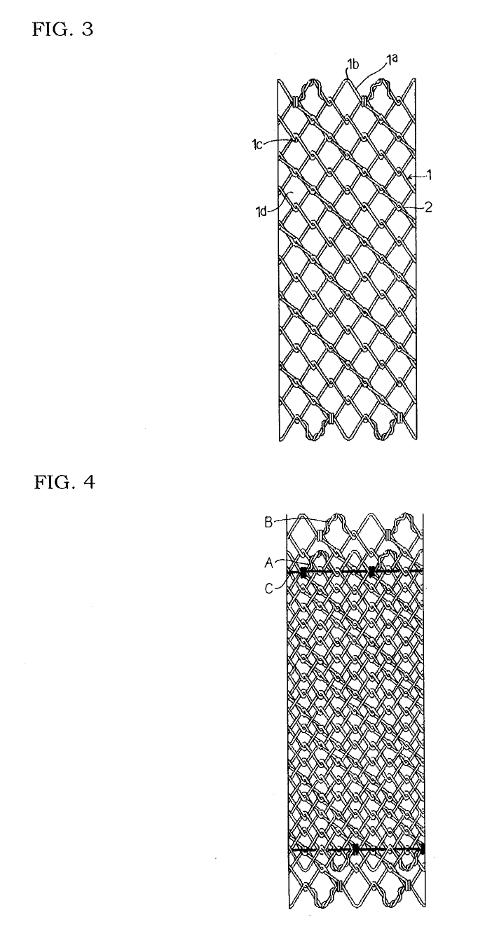

Looking at the structures of the inner stent and the outer stent, as shown in FIG. 3, the inner stent A and the outer stent B have a plurality of turns made in zigzags, so that the first shape memory wire 1 makes a plurality of straight-line portions 1a and the peak portions 1b and valley portions 1c that connect said straight-line portions 1a by a plurality of bending points. The valley portion of any one turn is twined to be connected with the peak portion 1d corresponding to the turn adjacent to this one turn to form a plurality of space portions 1d. In the diagonal direction, the second shape memory wire 2 has a structure twined with the first shape memory alloy wire 1 at a given interval.

FIG. 3 is a schematic view showing the structure of the inner stent A and outer stent B composing the present invention.

These inner stent A and outer stent B can be manufactured by first preparing a stent of a zigzag structure with the first shape memory alloy wire 1, as shown in (a) and (b) of FIG. 5, then twining the second shape memory alloy wire 2 with the first shape memory alloy wire 1 at a given interval along the diagonal direction.

FIG. 5(a) and (b) are schematic views showing the steps of manufacturing the inner stent A and the outer stent B.

The stent of the present invention excels in form recoverability (form conserving property) for maintaining the original form as it is.

Also, in the stent of the present invention, the outer stent B is inserted over the inner stent A, so that the outer surface of the inner stent and the inner surface of the outer stent are in close contact with each other, and the space portions of the inner stent and the space portions of the outer stent are arrayed alternately with each other. As a result, the space portions of the stents are characterized by having the unit sizes reduced.

The present invention, as described above, has the unit sizes of the space portions reduced consequently, so it can effectively prevent cancer cells from penetrating into the lumen.

Both ends of the inner stent A and the outer stent B are maintained in a condition fixed as one unit by the fixing threads C.

### INDUSTRIAL APPLICABILITY

The stent for intraluminal expansion of the present invention is used to keep the lumen expanded in case the esophagus is stenosed due to esophageal cancer, blood is not circulated smoothly due to arteriosclerosis, or the track for bile coming out from liver to flow is stenosed.

Although the present invention has been described in connection with the exemplary embodiments illustrated in the drawings, it is only illustrative. It will be understood by those skilled in the art that various modifications and equivalents can be made to the present invention. Therefore, the true technical scope of the present invention should be defined by the appended claims.

What is claimed is:

1. A stent for intraluminal expansion comprising an inner stent A, an outer stent B and fixing threads C for fixing these stents as one unit, wherein said outer stent B is inserted over said inner stent A in such a way that the space portions of said inner stent A and the space portions of said outer stent B are alternated with each other, so the outer surface of said inner stent and the inner surface of said outer stent are in close contact with each other, and both ends of said outer stent and inner stent are fixed as one unit by fixing threads C.

2. The stent of claim 1, further comprising a cylindrical film membrane inserted between the inner stent A and the outer stent B.

3. The stent of claim 2, wherein the cylindrical film membrane is composed of silicon resin.

4. The stent of claim 2, wherein only both of the ends of the inner stent A and the outer stent B are fixed as one unit by fixing threads C.

5. The stent of claim 1, wherein the inner stent A and the outer stent B have a plurality of turns made in zigzags so that the first shape memory alloy wire 1 makes a plurality of straight-line portions 1a and the peak portions 1b and valley portions 1c that connect said straight-line portions 1a by a plurality of bending points; the valley portion of any one turn is twined to be connected with the peak portion 1d corresponding to the turn adjacent to this one turn to form a plurality of space portions 1d; and in the diagonal direction the second shape memory alloy wire 2 is twined with said first shape memory wire 1 at a given interval.
